(19) United States
(12) Patent Application Publication
(10) Pub. No.: US 2004/0136940 A1
(43) Pub. Date: Jul. 15, 2004

(54) Cleaner compositions

(76) Inventor: Virginia Lazarowitz, Hatfield, PA (US)

Correspondence Address: COGNIS CORPORATION, PATENT DEPARTMENT, 300 BROOKSIDE AVENUE, AMBLER, PA, 19002, US

(21) Appl. No.: 10/696,250

(22) Filed: Oct. 29, 2003

Related U.S. Application Data
(60) Provisional application No. 60/422,570, filed on Oct. 31, 2002.

(51) Int. Cl. A61K 7/06; A61K 7/11; A61K 7/075; A61K 7/08

(52) U.S. Cl. 424/70.13; 424/70.21; 424/70.31

(57) ABSTRACT

Aqueous-based compositions useful as body cleansers comprised of the following components:

A) from about 1 to about 40% by weight of at least one nonionic alkyl polyglycoside surfactant or mixture of this surfactant with an anionic surfactant;

B) from about 0.5 to about 15% by weight of at least one glycerol fatty acid ester;

C) from about 0.25 to about 45% by weight of at least one alkoxylated glycerol fatty acid ester;

D) from 0 to about 10% by weight of a thickening agent;

E) from 0 to about 20% by weight of an amphoteric surfactant;

F) from 0 to about 10% by weight of a cationic surfactant;

G) from 0 to about 10% by weight of a nonionic surfactant other than an alkyl polyglucoside; and

H) water; provided that the composition contains at least about 25% by weight of water, and that when component A) includes an anionic surfactant, component F) is either not present or is a cationic surfactant which is compatible with the anionic surfactant; and wherein the percentages by weight are based in the total weight of the composition.

### CROSS-REFERENCE TO RELATED APPLICATIONS

This application claims the benefit of copending provisional application Serial No. 60/422,570 filed Oct. 31, 2002.

### FIELD OF THE INVENTION

This invention relates to body cleanser compositions; to methods for their preparation; and to their use.

### BACKGROUND OF THE INVENTION

Moisturizing body cleanser compositions typically contain large quantities of an oil such as soybean oil, maleated soybean oil, palm kernel oil, sunflower seed oil, or petrolatum.

Such body cleansers have a greasy feel and somewhat limited skin refatting capabilities.

### SUMMARY OF THE INVENTION

Other than in the operating examples, or where otherwise indicated, all numbers expressing quantities of ingredients or reaction conditions used herein are to be understood as modified in all instances by the term “about”.

This invention relates to aqueous-based compositions that are useful as body cleansers and which provide comparable skin feel to body cleansers containing large quantities of oil. The present compositions also provide excellent skin refatting, do not have a greasy feel, and provide high foam volumes during use.

It has surprisingly been found that moisturizing body cleansers can be formulated which are free of conventional oil components. Alkoxylated glycerol fatty acid esters impart immediate refatting effect onto a user's skin, whereas the glycerol fatty acid esters have surprisingly been found to impart a more long term refatting/moisturizing effect. Consequently, the combination of glycerol fatty acid esters and alkoxylated glycerol fatty acid esters provides both short- and long-term moisturizing effects without the use of any greasy-feeling oils which, over time, build up on the skin and yield an unpleasant feeling.

The other advantage associated with the cleansing compositions of the present invention is that they are self-emulsifiable, thereby eliminating the need for using conventional emulsifiers.

The aqueous-based compositions of the invention are comprised of the following components:

| Component | % by weight |
|---|---|
| A) nonionic surfactant or | 1 to 40, preferably 5 to 40, |
| nonionic/anionic mixtures | more preferably 10 to 35 |
| B) glycerol fatty acid ester or cetyl | 0.5 to 15, preferably 1 to 13, |
| palmitate or a mixture of the | more preferably 1 to 12 |
| foregoing in any proportion | |
| C) alkoxylated glycerol fatty | 0.25 to 45, preferably 0.5 to 10, |
| acid ester | more preferably 0.5 to 1.5 |
| D) thickening agent | 0 to 10, preferably 0.1 to 2 |
| E) amphoteric surfactant | 0 to 20, preferably 0.1 to 10 |
| F) cationic surfactant | 0 to 10, preferably 0.1 to 5 |
| G) nonionic surfactants other | 0 to 10, preferably 0.1 to 5 |
| than component A) | |
| H) water | remainder, provided that the |
| | compositions contain at least |
| | 25% by weight of water |

It should be noted that when component A) includes an anionic surfactant, component F) is either not present or is a cationic surfactant which is compatible with the anionic surfactant. Such compatible cationic surfactants include the polyquaternium surfactants, e.g. polyquaternium 7, polyquaternium 16, and polyquaternium 52.

### DETAILED DESCRIPTION OF THE INVENTION

The nonionic surfactant of component A) is one or more alkyl polyglycosides having the formula I below:

R₁O(R₂O)_{b}(Z)ₐ (1)

wherein R₁ is a monovalent organic radical having from about 6 to about 30 carbon atoms, preferably from 6 to 22 carbon atoms, and more preferably having an average of from 10 to 18 carbon atoms; R₂ is a divalent alkylene radical having from 2 to 4 carbon atoms; Z is saccharide residue having 5 or 6 carbon atoms; b is a number having a value from 0 to about 12, a is a number having a value from 1 to about 6, preferably from 1.2 to 2.2, and more preferably from 1.4 to 1.5. Preferred alkyl polyglycosides which can be used in the compositions according to the invention have the formula I wherein Z is or includes a glucose residue. Such alkyl polyglycosides are commercially available, for example as APG®, GLUCOPON®, or PLANTAREN® surfactants from Cognis Corporation, Ambler, Pa., 19002. Examples of such surfactants include but are not limited to:

1. GLUCOPON® 225DK Surfactant—an alkyl polyglycoside in which the alkyl group contains 8 to 10 carbon atoms and having an average degree of polymerization of 1.7.

2. GLUCOPON® 425N Surfactant—an alkyl polyglycoside in which the alkyl group contains 8 to 16 carbon atoms, having an average of 10.3 carbon atoms, and having an average degree of polymerization of 1.5.

3. GLUCOPON® 625UP Surfactant—an alkyl polyglycoside in which the alkyl group contains 12 to 16 carbon atoms and having an average degree of polymerization of 1.6.

4. APG® 325N Surfactant—an alkyl polyglycoside in which the alkyl group contains 9 to 11 carbon atoms and having an average degree of polymerization of 1.5. 5. GLUCOPON®) 600UP Surfactant—an alkyl polyglycoside in which the alkyl group contains 12 to 16 carbon atoms and having an average degree of polymerization of 1.4.

6. PLANTAREN® 2000 Surfactant—a C₈-C₁₋₆ alkyl polyglycoside in which the alkyl group contains 8 to 16 carbon atoms and having an average degree of polymerization of 1.5.

7. PLANTAREN® 1300 Surfactant—a C₁₂-C₁₆ alkyl polyglycoside in which the alkyl group contains 12 to 16 carbon atoms and having an average degree of polymerization of 1.6.

8. GLUCOPON® 220N Surfactant—an alkyl polyglycoside in which the alkyl group contains 8 to 10 carbon atoms and having an average degree of polymerization of 1.5.

Other examples of alkyl polyglycosides that can be used herein include alkyl polyglycoside surfactants which are comprised of mixtures of compounds of formula I wherein Z represents a moiety derived from a reducing saccharide containing 5 or 6 carbon atoms; a is a number having a value from 1 to about 6; b is zero; and R₁ is an alkyl radical having from 8 to 20 carbon atoms. The compositions are characterized in that they have increased surfactant properties and an HLB in the range of about 10 to about 16 and a non-Flory distribution of glycosides, which is comprised of a mixture of an alkyl monoglycoside and a mixture of alkyl polyglycosides having varying degrees of polymerization of 2 or higher in progressively decreasing amounts, in which the amount by weight of polyglycoside having a degree of polymerization of 2 and mixtures thereof with the polyglycoside having a degree of polymerization of 3 predominate in relation to the amount of monoglycoside, said composition having an average degree of polymerization of about 1.8 to about 3. Such compositions, also known as peaked alkyl polyglycosides, can be prepared by separation of the monoglycoside from the original reaction mixture of alkyl monoglycoside and alkyl polyglycosides after removal of the alcohol. This separation may be carried out by molecular distillation and normally results in the removal of about 70-95% by weight of the alkyl monoglycosides. After removal of the alkyl monoglycosides, the relative distribution of the various components, mono- and poly-glycosides, in the resulting product changes and the concentration in the product of the polyglycosides relative to the monoglycoside increases as well as the concentration of individual polyglycosides to the total, i.e. DP2 and DP3 fractions in relation to the sum of all DP fractions. Such compositions are disclosed in U.S. Pat. No. 5,266,690, the entire contents of which are incorporated herein by reference.

The most preferred alkyl polyglycoside for use in the present invention is PLANTAREN® 2000.

When the alkyl glycosides are used in combination with one or more anionic surfactants in component A), the ratio of anionic surfactant to alkyl glycoside is preferably in the range of from 2:1 to 6:1.

The compatible anionic surface active agents include those surface active or detergent compounds which contain an organic hydrophobic group containing generally 8 to 26 carbon atoms and preferably 10 to 18 carbon atoms in their molecular structure, and at least one water-solubilizing group selected form the group of sulfonate, sulfate, carboxylate, phosphonate and phosphate so as to form a water-soluble surfactant.

Examples of suitable anionic surfactants which fall within the scope of the anionic surfactant class include the water-soluble salts, e.g., the sodium, ammonium, and alkanolammonium, e.g. ethanolammonium salts, of higher fatty acids or resin salts containing about 8 to 20 carbon atoms, preferably 10 to 18 carbon atoms. Suitable fatty acids can be obtained from oils and waxes of petroleum or animal or vegetable origin, e.g., tallow, grease, coconut oil, tall oil, palm oil, and mixtures thereof. Particularly useful are the sodium and potassium salts of the fatty acid mixtures derived from coconut oil and tallow.

The anionic class of surfactants also includes the water-soluble sulfated and sulfonated synthetic surfactants having an alkyl radical of 8 to 26, and preferably 12 to 22 carbon atoms, in their molecular structure, as well as sulfosuccinates. Also, the compounds formed by including one to 40 moles of ethylene oxide in the foregoing surfactants can also be used.

The anionic surfactants also include C₈-C₁₈ acyl sarcosinates (e.g. sodium lauroyl sarcosinate), sodium and potassium salts of the reaction product of higher fatty acids containing 8-18 carbon atoms in the molecule esterified with isethionic acid, and sodium and potassium salts of the C₈-C₁₈ acyl N-methyl taurides, e.g., sodium cocoyl methyl taurate and potassium stearoyl methyl taurate.

Anionic phosphate surfactants in which the anionic solubilizing group is an oxyacid of phosphorous are also useful in the present compositions. Suitable phosphate surfactants are the sodium, potassium and ammonium alkyl phosphate esters. The compounds formed by including about one to 40 moles of ethylene oxide in the foregoing esters are also satisfactory.

Other anionic surfactants include carboxylates of alkyl polyglycosides and other anionic derivatives thereof.

The component B) glycerol fatty acid esters can be one or more of a glycerol mono- or di-ester, in which the fatty acid moiety can contain from 6 to 30 carbon atoms, preferably from 12 to 22, and more preferably from 12 to 18 carbon atoms. The fatty acid moiety can be saturated or olefinically unsaturated. The preferred glycerol fatty acid ester is glycerol monooleate.

The alkoxylated glycerol fatty acid esters of component C) include alkoxylated glycerol having from 1 to 50 alkyleneoxy groups which can be ethyleneoxy, propyleneoxy, and butyleneoxy groups, or mixtures thereof, preferably all ethyleneoxy groups, wherein the alkoxylated glycerol is esterified with a saturated or unsaturated fatty acid or esterifiable derivative thereof having the carbon atom ranges given above for component B). PEG 7 glycerol monococoate is preferred for use herein. The term PEG means polyethyleneglycol, and the number 7 means the number of such units present in the molecule.

The component D) thickening agent can be any pharmacologically compatible thickening agent or mixture of such agents, including, but not limited to, guar hydroxypropyl triammonium chloride, a polyacrylic acid polymer, sodium carboxymethyl-cellulose, ethoxylated cellulose, hydroxy-propylcellulose, hydroxyethyl cellulose, hydroxyethyl stearyl amide, ethylene glycol monostearate, stearic diethanolamide, coconut fatty acid diethanolamide, lauric diethanolamide, lauric/myristic diethanolamide, ethylene glycol distearate, n-octadecanol, lauric monoisopropanolamide, PEG (400-1000) mono- or di-stearates, glycerol dioleate, alkali metal alginates, xanthan gum, and the like. Guar hydroxypropyl triammonium chloride and polyacrylic acid polymers are preferred for use herein.

The amphoteric surfactants of component E) contain both an acidic and basic hydrophilic group. Ether or hydroxyl groups may also be present to enhance the hydrophilicity of the surfactant molecule. Examples of amphoteric surfactants include amino acids and their derivatives in which the nitrogen atom tends to become protonated with decreasing pH of the solution. Amino acid salts, under these conditions, contain both a positive and a negative charge on the same molecule.

Also included are water-soluble salts of derivatives of aliphatic amines which contain at least one cationic group, e.g. non-quaternary nitrogen, quaternary ammonium or quaternary phosphonium group, at least one alkyl group of 8-18 carbon atoms and may be straight chain or branched and the specific cationic atom may be part of a heterocyclic ring. Amphoteric imidazolinium derivatives are prepared from the 2-alkyl-1 (2-hydroxyethyl)-2-imidazolines and from sodium chloroacetate.

Further examples are betaines and sulfobetaines, such as cocoamidopropyl betaine; amphoacetates such as cocoamphodiacetate; monosodium N-lauryl-β-iminodipropionic acid; and the like.

The cationic surfactants of component F) include normal primary amines RNH₂ wherein R is C₁₂-C₁₅; the diamines such as those of the type RNHC₂H₄NH₂ wherein R is an alkyl group of about 12 to 22 carbon atoms, such as N-2-aminoethyl stearyl amine and N-2-aminoethyl myristyl amine; amide-linked amines such as those of the type R₁CONHC₂H₄NH₂ wherein R₁ is an alkyl group of about 8 to 20 carbon atoms, such as N-2-amino ethylstearyl amide and N-amino ethylmyristyl amide; quaternary ammonium compounds wherein typically one of the groups linked to the nitrogen atom is an alkyl group of about 8-22 carbon atoms and three of the groups linked to the nitrogen atom are alkyl groups which contain 1 to 3 carbon atoms, including alkyl groups bearing inert substituents, such as phenyl groups, and there is present an anion such as halogen, acetate, methosulfate, etc. The alkyl group may contain intermediate linkages such as amido which do not substantially affect the hydrophobic character of the group, e.g. stearyl amido propyl quaternary ammonium chloride. Typical quaternary ammonium detergents are ethyldimethyl-stearyl ammonium chloride, benzyl-dimethyl-stearyl ammonium chloride, trimethyl-stearyl ammonium chloride, trimethyl-cetyl ammonium bromide, dimethyl-ethyl-lauryl ammonium chloride, dimethyl-propyl-myristyl ammonium chloride, and the corresponding methosulfates and acetates.

Also included are the so-called ester quats and polyquaternium compounds.

The nonionic surfactants of component G) which are other than the alkyl polyglycosides of component A) include polyoxyethylene-solubilized nonionics, which can be terminated with less water soluble groups such as polyoxypropylene and methyl groups.

Also included are the condensation products of an organic aliphatic or alkyl aromatic hydrophobic compound and hydrophilic ethylene oxide groups. Practically any hydrophobic compound having a carboxy, hydroxy, amido, or amino group with a free hydrogen attached to the nitrogen can be condensed with ethylene oxide or with the polyhydration product thereof, polyethylene glycol, to form a nonionic detergent. Further, the length of the polyethyleneoxy chain can be adjusted to achieve the desired balance between the hydrophobic and hydrophilic elements.

The nonionic detergents include the polyethylene oxide condensate of one mole of alkyl phenol containing from about 6 to 12 carbon atoms in a straight- or branched-chain configuration with about 5 to 30 moles of ethylene oxide, e.g., nonyl phenol condensed with 9 moles of ethylene oxide, dodecyl phenol condensed with 15 moles of ethylene oxide. Condensation products of the corresponding alkyl thiophenols with 5 to 30 moles of ethylene oxide are also suitable.

Still other suitable nonionics are the polyoxyethylene polyoxypropylene adducts of 1-butanol. The hydrophobe of these nonionics has a minimum molecular weight of 1,000 and consists of an aliphatic monohydric alcohol containing from 1 to 8 carbon atoms to which is attached a chain of oxyethylene and oxypropylene. The weight ratio of oxypropylene to oxyethylene covers the range of 95:5 to 85:15. Attached to this is the hydrophilic polyoxyethylene chain which is from 44.4 to 54.6 percent of the total molecular weight of 1,400 to 4,000.

Also included in the nonionic detergent class are the condensation products of a higher alcohol containing about 8 to 18 carbon atoms in a straight or branched-chain configuration condensed with about 5 to 30 moles of ethylene oxide, e.g., lauryl-myristyl alcohol condensed with about 16 moles of ethylene oxide.

Other suitable nonionics may be derived by the condensation of ethylene oxide with the product resulting from the reaction of propylene oxide and ethylene diamine. The molecular weight varies from 500 to 4,500 or more.

Carboxylic acid esters are also suitable. In the carboxylic acid ester series of surfactants, the hydrophobe, a naturally occurring fatty acid, is solubilized with the hydroxyl groups of polyols or the ether and terminal hydroxyl groups of ethylene oxide chains. Included in this group are glycerol esters; polyoxyethylene esters; and hydrosorbitol esters; ethoxylated anhydrosorbitol esters; natural ethoxylated fats, oils, and waxes; and glycol esters of fatty acids.

Carboxylic amide nonionic surfactants are condensation products of fatty acids and hydroxyalkyl amines. They include diethanolamine condensates, monoalkanolamine condensates, and polyoxyethylene fatty acid amides.

Fatty acyl glucamides (FAGA) or polyhydroxyamides (PHA) can also be used. FAGA is produced via reaction of fatty acid methyl ester with N-methyl glucamine and attendant elimination of methanol. The methyl ester is produced via the standard route of transesterification with fatty triglycerides; the glucamine, via reaction between glucose and methylamine with attendant hydrogenation and elimination of water.

In addition to the above components (A through F) of the aqueous-based compositions of the invention, other components can optionally be present as well, e.g. emollients such as dicebrylyl carbonate and/or dicebrylyl ether.

The compositions of the invention can be readily prepared by mixing the components together with or without moderate heating.

The invention will be illustrated but not limited by the following examples.

### EXAMPLES

### Example 1

The following body lotion composition was prepared by mixing together the following components at room temperature:

| Component | % by weight |
|---|---|
| PLANTAREN ® 2000¹ | 34.00 |
| LAMESOFT ® PO-65² | 3.00 |
| CETIOL ® HE³ | 0.50 |
| CARBOPOL ® 2020⁴ | 2.00 |
| polyquaterternium 10⁵ | 0.50 |
| LAMESOFT ™ BENZ⁶ | 3.00 |
| VELVETEX ® BA-35⁷ | 10.00 |
| water | 47.00 |
| | 100.00 |

¹a C₁₂—C₁₆ alkyl polyglucoside having an average degree of polymerization of 1.6.
²cocoglucoside and glycerol monooleate in a 2:1 weight ratio.
³PEG 7 glycerol cocoate.
⁴a polyacrylic acid polymer.
⁵a polyquaternium cationic surfactant.
⁶a mixture of glycol distearate, coco glucoside, glyceryl oleate, glyceryl stearate.
⁷cocoamidopropyl betaine.

### Example 2

The following body lotion composition was prepared by mixing together the following components at room temperature:

| Component | % by weight |
|---|---|
| PLANTAREN ® 2000 | 34.00 |
| LAMESOFT ® PO-65 | 3.00 |
| CETIOL ® HE | 0.50 |
| CARBOPOL ® 2020 | 2.00 |
| polyquaterternium 10 | 0.50 |
| LAMESOFT ™ BENZ | 5.00 |
| DERIPHAT ® 106 C⁸ | 10.00 |
| water | 45.00 |
| | 100.00 |

⁸a 3% active aqueous solution of monosodium n-lauryl betaimido-dipropionic acid

### Example 3

The compositions of Example 1 and 2 were sensory evaluated by 25 panelists and compared to the commercial product Oil of Olay™. The results are set forth in Table 1 below:

TABLE 1

| | |
|---|---|
| Example 1 composition | Example 2 composition |
| vs. Oil of Olay ™ - | vs. Oil of Olay ™ - |
| percentage of panel | percentage of panel |
| testers who considered | testers who considered |
| the Example 1 composition | the Example 2 composition |
| to be equal or better than | to be equal or better than |
| Oil of Olay ™ | Oil of Olay |

| | | | |
|---|---|---|---|
| soapy | 56% | soapy | 83% |
| after rinse | 64% | after rinse | 83% |
| after drying | 60% | after drying | 83% |

### Example 4

The compositions of Examples 1 and 2 were tested for foam levels in a blender and with the use of a scrunchie (a nylon poof used to apply body washes). For comparison purposes, several commercial products were also tested. The results are set forth in Table 2 below:

TABLE 2
In this table the numbers represent the milliliters of foam generated.

| | |
|---|---|
| Blender | Scrunchie with 0.5 g |

| Product | without sebum | of artificial sebum* | |
|---|---|---|---|
| Oil of Olay ™ | 270 | 250 | 1000 |
| Bath and Body Oil | 630 | 370 | 1200 |
| Olay ™ Daily | 380 | 250 | 600 |
| Dove ™ | 420 | 260 | 1200 |
| Example 1 | 500 | 300 | 1200 |
| Example 2 | 540 | 320 | 1200 |

*supplied by Scientific Services Corporation

What is claimed is:

1. An aqueous-based composition useful as a body cleanser comprised of the following components:
A) from about 1 to about 40% by weight of at least one nonionic alkyl polyglycoside surfactant or mixture of nonionic alkyl polyglycoside with an anionic surfactant;
B) from about 0.5 to about 15% by weight of at least one glycerol fatty acid ester;
C) from about 0.25 to about 45% by weight of at least one alkoxylated glycerol fatty acid ester;
D) up to about 10% by weight of a thickening agent;
E) up to about 20% by weight of an amphoteric surfactant;
F) up to about 10% by weight of a cationic surfactant;
G) up to about 10% by weight of a nonionic surfactant other than an alkyl polyglucoside; and
H) at least about 25% by weight of water, all weights being based on the total weight of the composition, and with the proviso that when component A) includes an anionic surfactant, component F) is either not present or is a cationic surfactant compatible with an anionic surfactant.

2. The composition of claim 1 wherein component A) is a nonionic alkyl polyglycoside.

3. The composition of claim 1 wherein component A) is a mixture of a nonionic alkyl polyglycoside and an anionic surfactant.

4. The composition of claim 1 wherein component A) is present in an amount of from about 5 to about 40% by weight; component B) is present in an amount of from about 1 to about 13% by weight; and component C) is present in an amount of from about 0.5 to about 10% by weight.

5. The composition of claim 1 wherein component A) is present in an amount of from about 10 to about 35% by weight; component B) is present in an amount of from about 1 to about 12% by weight; and component C) is present in an amount of from about 0.5 to about 1.5% by weight.

6. The composition of claim 1 wherein component B) is a glycerol mono- or di-ester having a fatty acid moiety containing from about 6 to 30 carbon atoms.

7. The composition of claim 1 wherein component C) is an ethoxylated glycerol having from 1 to about 50 ethyleneoxy groups and which is esterified with a saturated or unsaturated fatty acid or esterifiable derivative thereof containing from about 6 to 30 carbon atoms.

8. The composition of claim 1 wherein component A) is a nonionic alkyl polyglycoside surfactant; component B) is a glycerol mono- or di-ester having a fatty acid moiety containing from about 6 to 30 carbon atoms; and component C) is an ethoxylated glycerol having from 1 to 50 ethyleneoxy groups and which is esterified with a saturated or unsaturated fatty acid or esterifiable derivative thereof containing from about 6 to 30 carbon atoms.

9. The composition of claim 1 wherein the composition is free of any oil components other than those of components B) and C).

10. An aqueous-based composition useful as a body cleanser comprised of the following components:
A) from about 10 to about 35% by weight of an alkyl polyglycoside;
B) from about 1 to about 12% by weight of glycerol monooleate;
C) from about 0.5 to about 1.5% by weight of PEG 7 glycerol monococoate; and
D) remainder, water.

11. The composition of claim 10 wherein component A) is present in an amount of from about 34% by weight; component B) is present in an amount of from about 1% by weight; and component C) is present in an amount of from about 0.5% by weight.

12. The composition of claim 10 wherein the composition is free of oil components other than those of components B) and C).

13. A process for simultaneously washing and moisturizing skin comprising contacting the skin with a body cleanser composition containing:
A) from about 1 to about 40% by weight of at least one nonionic alkyl polyglycoside surfactant or mixture of nonionic alkyl polyglycoside with an anionic surfactant;
B) from about 0.5 to about 15% by weight of at least one glycerol fatty acid ester;
C) from about 0.25 to about 45% by weight of at least one alkoxylated glycerol fatty acid ester;
D) up to about 10% by weight of a thickening agent;
E) up to about 20% by weight of an amphoteric surfactant;
F) up to about 10% by weight of a cationic surfactant;
G) up to about 10% by weight of a nonionic surfactant other than an alkyl polyglucoside; and
at least about 25% by weight of water, all weights being based on the total weight of the composition, and with the proviso that when component A) includes an anionic surfactant, component F) is either not present or is a cationic surfactant compatible with an anionic surfactant.

14. The process of claim 13 wherein component A) is a nonionic alkyl polyglycoside.

15. The process of claim 13 wherein component A) is a mixture of a nonionic alkyl polyglycoside and an anionic surfactant.

16. The process of claim 13 wherein component A) is present in an amount of from about 5 to about 40% by weight; component B) is present in an amount of from about 1 to about 13% by weight; and component C) is present in an amount of from about 0.5 to about 10% by weight.

17. The process of claim 13 wherein component A) is present in an amount of from about 10 to about 35% by weight; component B) is present in an amount of from about 1 to about 12% by weight; and component C) is present in an amount of from about 0.5 to about 1.5% by weight.

18. The process of claim 13 wherein component B) is a glycerol mono- or di-ester having a fatty acid moiety containing from about 6 to 30 carbon atoms.

19. The process of claim 13 wherein component C) is an ethoxylated glycerol having from 1 to about 50 ethyleneoxy groups and which is esterified with a saturated or unsaturated fatty acid or esterifiable derivative thereof containing from about 6 to 30 carbon atoms.

20. The process of claim 13 wherein component A) is a nonionic alkyl polyglycoside surfactant; component B) is a glycerol mono- or di-ester having a fatty acid moiety containing from about 6 to 30 carbon atoms; and component C) is an ethoxylated glycerol having from 1 to 50 ethyleneoxy groups and which is esterified with a saturated or unsaturated fatty acid or esterifiable derivative thereof containing from about 6 to 30 carbon atoms.

21. The process of claim 13 wherein the composition is free of oil components other than those of components B) and C).
